(19) United States
(12) Patent Application Publication
(10) Pub. No.: US 2014/0358099 A1
(43) Pub. Date: Dec. 4, 2014

(54) RF Backscatter Sensor for Measuring Moisture and Other Biological Data

(71) Applicants: Gregory Durgin, Atlanta, GA (US); Blake Marshall, Atlanta, GA (US); Danielle Bradley, Atlanta, GA (US); Andrew Kirsch, Atlanta, GA (US)

(72) Inventors: Gregory Durgin, Atlanta, GA (US); Blake Marshall, Atlanta, GA (US); Danielle Bradley, Atlanta, GA (US); Andrew Kirsch, Atlanta, GA (US)

(73) Assignees: Children's Healthcare of Atlanta, Atlanta, GA (US); Georgia Tech Research Corporation, Atlanta, GA (US)

(21) Appl. No.: 14/291,458

(22) Filed: May 30, 2014

Related U.S. Application Data
(60) Provisional application No. 61/828,897, filed on May 30, 2013.

(51) Int. Cl. A61F 13/42; G08B 21/20; G01N 27/22

(52) U.S. Cl. CPC A61F 13/42; G01N 27/223; G08B 21/20; A61F 2013/424
USPC 604/361; 324/664; 340/573.5

(57) ABSTRACT

A moisture sensor unit for sending moisture in an environment includes a passive moisture sensor and two passive RFID tags. The two passive RFID tags are spaced apart from each other and disposed on opposite sides of the moisture sensor. Each of the RFID tags is electrically coupled to the moisture sensor and each of the RFID tags includes a circuit that determines an amount of moisture that is associated with the moisture sensor. Each of the RFID tags is configured to respond to a first radio frequency signal with a second radio frequency signal that includes information about the amount of moisture.

### CROSS-REFERENCE TO RELATED APPLICATION(S)

This application claims the benefit of U.S. Provisional Patent Application Ser. No. 61/828,897, filed May 30, 2013, the entirety of which is hereby incorporated herein by reference.

### BACKGROUND OF THE INVENTION

1. Field of the Invention

The present invention relates to sensing systems and, more specifically, to a system for sensing moisture.

2. Description of the Related Art

Incontinence is a common problem for children and adults with developmental disabilities. It is common for parents and caregivers to seek incontinence treatment for those under their care. Reasons for concern include financial burdens, social stigmatization, and strained parent-child interactions. The treatment of incontinence typically employs well established behavioral psychological interventions, which have been shown to be effective.

Specific treatment procedures for continence included regularly scheduled trips to the restroom, fluid loading, and positive reinforcement for remaining dry and for continent voids. One treatment protocol includes use of an alarm device to signal incontinence and practicing appropriate toileting behavior when accidents occur.

Devices to signal incontinence have proven useful in the immediate detection of a void. Often, these devices are attached to the child's undergarments and an alarm (sometimes accompanied by a vibration) is emitted to signal when a void begins to occur so that a parent or trainer can immediately take the child to the toilet. This sound can be effective because it may serve as a cue to the child that continent voids will receive a positive consequence such as praise or a toy (a common and effective behavioral strategy). Through the detection of a void as soon as it occurs, the trainer can respond immediately, making it more likely that continence will increase through the continuous and immediate provision of positive consequences. The use of these systems has been extended to the treatment of other toileting problems such as nighttime incontinence.

Currently used void alerting devices include a wire that attaches to a sensor in a user's undergarments and to an alarm. This wire can cause problems when used with children who engage in maladaptive behaviors such as aggression and self-injury. If a signaling device with a wire is used with a child who engages in such behaviors, significant physical consequences may occur (e.g., the wire may become wrapped around the child's body parts or the wire may be used as a tool for aggressive acts). Additionally, users may touch or manipulate the wire, which can result in disablement of the alarm.

Therefore, there is a need for a wireless incontinence alerting device that is operable while the user is in different positions.

### SUMMARY OF THE INVENTION

The disadvantages of the prior art are overcome by the present invention which, in one aspect, is a moisture sensor unit for sending moisture in an environment that includes a passive moisture sensor and two passive RFID tags. The two passive RFID tags are spaced apart from each other and disposed on opposite sides of the moisture sensor. Each of the RFID tags is electrically coupled to the moisture sensor and each of the RFID tags includes a circuit that determines an amount of moisture that is associated with the moisture sensor. Each of the RFID tags is configured to respond to a first radio frequency signal with a second radio frequency signal that includes information about the amount of moisture.

In another aspect, the invention is a sensing system for sensing moisture in an environment that includes a radio frequency transceiver, a passive moisture sensor and two passive RFID tags. The radio frequency transceiver is configured to transmit a first signal and to receive a second signal.

In yet another aspect, the invention is a diaper for absorbing moisture voided by a wearer, which includes an outer shell, an absorbent core structure and a moisture sensor unit. The absorbent core structure is disposed on the outer shell and is configured to absorb moisture. The moisture sensor unit is coupled to the absorbent core structure and includes a passive moisture sensor and two passive RFID tags. The passive moisture sensor is disposed so that at least one electrical property of the moisture sensor depends on an amount of moisture in the absorbent core structure. The two passive RFID tags are spaced apart from each other and disposed on opposite sides of the moisture sensor. Each of the RFID tags is electrically coupled to the moisture sensor. Each of the RFID tags includes a circuit that determines the amount of moisture in the absorbent core structure. Each of the RFID tags is configured to respond to a first radio frequency signal with a second radio frequency signal that includes information about the amount of moisture. A first one of the two passive RFID tags is disposed in a first portion of the diaper so that if a wearer of the diaper is facing upwardly, then the a first one of the two passive RFID tags will be in a position to be responsive to the first signal. The second one of the two passive RFID tags is disposed in a second portion of the diaper, different from the first portion of the diaper, so that if the wearer of the diaper is facing downwardly, then the a second one of the two passive RFID tags will be in a position to be responsive to the first signal.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a schematic diagram of one embodiment of a moisture sensor unit.

FIG. 1B is a schematic diagram of the embodiment shown in FIG. 1A employed in a diaper.

FIG. 2 is a schematic diagram of one detailed embodiment of a moisture sensing unit.

FIG. 3 is a schematic diagram of one embodiment of a moisture sensing system.

FIG. 4 is a block diagram showing components employed in a moisture sensing unit.

FIG. 5 is a flow chart showing one method of operating a moisture sensing unit.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Also, as used herein, “global computer network” includes the Internet.

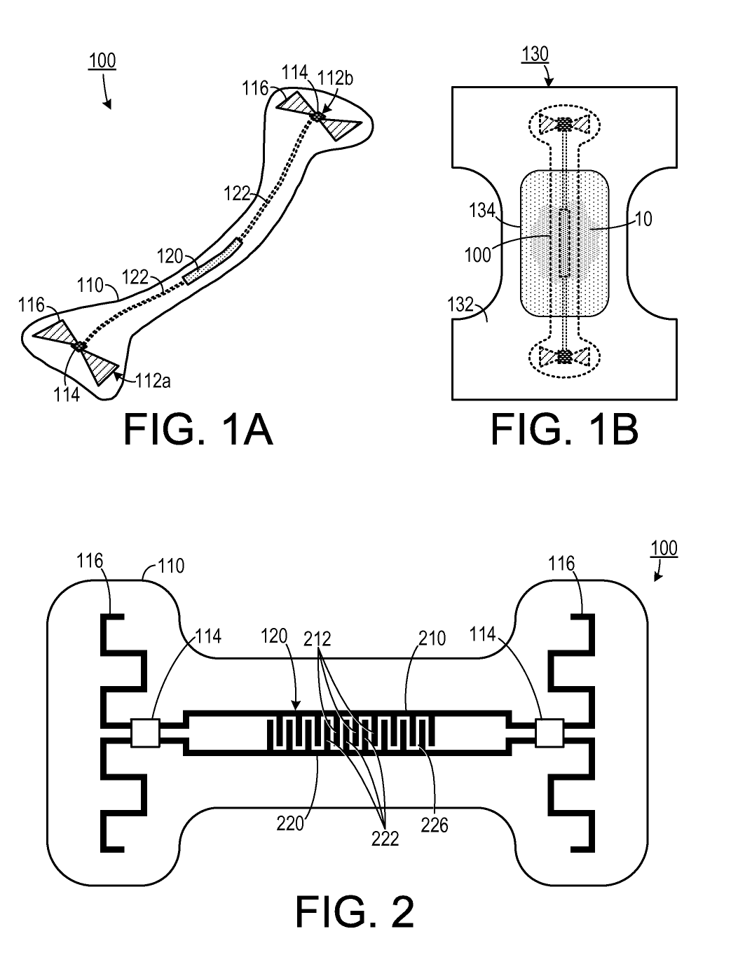

As shown in FIGS. 1A and 1B, one example of a moisture sensor unit 100 for sensing moisture in an environment, such as a diaper 130. (It should be readily appreciated that the sensing unit 100 could be used to sense moisture in many other environments, including soil containers, food items, etc.) The sensing unit 100 includes a passive moisture sensor 120 that is coupled to a first passive RFID tag 112a and an oppositely disposed second passive RFID tag 112b through conductive traces 122. Each passive RFID tag 112a and 112b includes an RFID antenna structure 116 and a circuit 114 coupled to both the antenna structure 116 and the conductive trace 122. In many embodiments, a flexible waterproof substrate 110 envelops the passive moisture sensor 120, the conductive traces 122 and the passive RFID tags 112a and 112b.

The circuit 114 is responsive to the moisture sensor 120 and calculates the amount of moisture sensed by the moisture sensor. The circuit 114 also generates a signal, such as a digital signal, that is representative of the amount of moisture 10. When queried, the RFID tags RFID tags 112a and 112b respond with a radio frequency signal upon which the digital signal is modulated.

The diaper 130 embodiment, as shown in FIG. 1B, includes an outer shell 132, which is typically made of a plastic film, and an absorbent core structure 134 disposed thereon, which typically includes cellulose and a super absorbent polymer disposed under a non-woven fabric that absorbs moisture 10. The sensing unit 100 can be disposed between the outer shell 132 and the core structure 134. The first RFID tag 112a is disposed in the front portion of the diaper 130 so that if a wearer of the diaper is facing upwardly, then the first RFID tag 112a will be able to respond to a querying radio frequency signal. The second RFID tag 112b is disposed in the back portion of the diaper 130 so that if the wearer of the diaper is facing downwardly, then the a second RFID tag 112b will be able to respond to a querying radio frequency signal. In this way, there is always an RFID tag 112a and 112b that can respond to a querying signal, irrespective of the position of the wearer.

As shown in FIG. 2, in one embodiment, the sensing unit 100 employs circuit elements that are printed on a surface of the substrate 110 using a conductive ink and well known printing methods (such as, for example, ink jet printing and screen printing). In this embodiment, the moisture sensor 120 includes a first conductive strip 210 from which a first plurality of spaced apart conductive fingers 212 extend. A second conductive strip 220 has a second plurality of spaced apart conductive fingers 222 extending transversely therefrom. The second conductive strip 220 disposed opposite to the first conductive strip 210 so that the second plurality of spaced apart conductive fingers 222 are interleaved with the first plurality of spaced apart conductive fingers 212. A a non-conductive path 226 is defined between the first conductive strip 210 and the second conductive strip 220 so that they act as a capacitor. The electrical permittivity the moisture sensor 120 is a function of the amount of moisture in the environment around the first conductive strip 210 and the second conductive strip 220. Thus, the capacitance of the moisture sensor 120 is a function of the amount of moisture in the environment adjacent thereto.

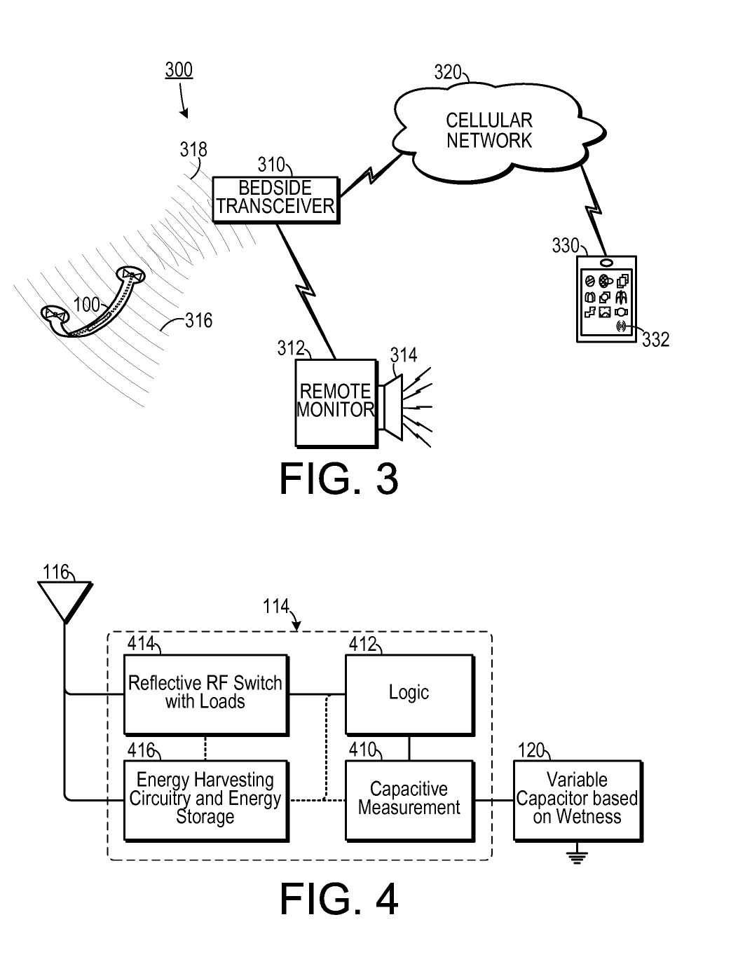

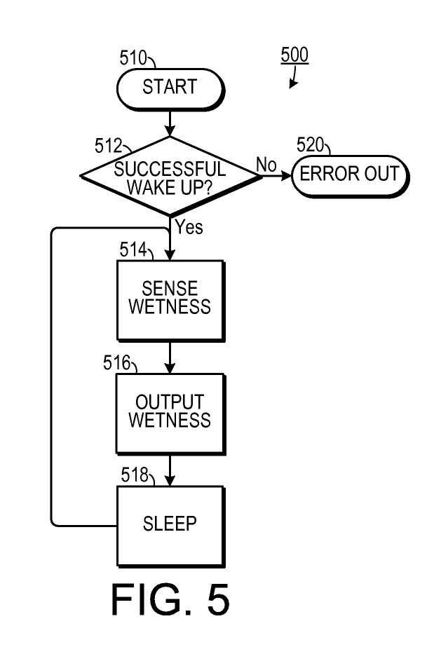

As shown in FIG. 3, one embodiment of a sensing system 300 for sensing moisture in an environment includes a moisture sensing unit 100 that is responsive to a radio frequency transceiver 310. The radio frequency transceiver 310 (such as a bedside transceiver when used in a patient care scenario) generates a querying first signal 316 that is received by the moisture sensing unit 100. The moisture sensing unit 100 reflectively responds to first signal 316 with a responsive second signal 318 that contains information modulated thereon that indicates the amount of moisture in the environment. The transceiver can include circuitry (e.g., a processor) that evaluates the moisture information and takes an action when the amount of moisture exceeds a predefined threshold. For example, the transceiver 310 could transmit a signal to a remote monitor 312 which sets off an audible alarm 314 that is perceptible by an interested person (such as a caregiver). Similarly, the transceiver 310 could transmit a signal to a network 320 (such as a cellular network or a global computer network), which could notify an interested person via a cellphone 330 running a notification application 332.

The use of reflected radio wave data transmission makes it possible for the sensing unit 100 to be less intrusive, safer and more effective than previous system. Such reflected radio wave data transmission allows the sensors to use extraordinarily low power levels to communicate with a wireless access point using reflection-based radios. As a result, the embodiment shown herein does not require batteries, but instead harvests energy from the transceiver's 310 own radio transmissions. Without the need for a battery, the wireless sensor 100 is small, low-cost, and non-intrusive.

Circuit 114, in one embodiment, may include an energy harvesting and storage unit 416 that harvests energy from the first signal 316 with which it powers the rest of the system. A reflective radio frequency switch 414 receives the first signal 316 and transmits the second signal 318 to the RFID antenna 116. Upon receipt of the first signal 316, the switch 414 activates a logic unit 412 (which could include a microcontroller, a programmable logic array, a specifically designed logic circuit, etc.) that engages a capacitive measurement circuit 410. The capacitive measurement circuit 410 is responsive to the moisture sensor 120, which behaves as a variable capacitor having a capacitance that is a function of moisture in the environment. The logic unit 412 transmits a digital indication of the amount of wetness to switch 414, which modulates this information onto the second signal 318, which is essentially a modified reflected version of the first signal 316. In such an embodiment, no new carrier wave is generated and the circuit 114 does not require an oscillator or a complete transmitter. Modulation can be done by one of many commonly known modulating methods, including phase modulation (e.g., wherein a “0” is represented by a zero phase shift and a “1” is represented by a 180° phase shift), amplitude modulation, etc. The system can be configured to report the actual concentration of moisture, rather than just the presence of moisture.

In one embodiment, the logic unit 412 operates according to the following method 500: It starts 510 upon receiving the first signal 316 and performs a self-test 512 to determine if it has activated successfully. If not, it follows a predetermined error action 520. Upon successful activation, it senses the wetness of the environment 514 and outputs data 516 indicative of the amount of wetness. The logic unit 412 then returns to a sleep state 518 until the next querying signal is received.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A moisture sensor unit for sending moisture in an environment, comprising:
(a) a passive moisture sensor; and
(b) two passive RFID tags that are spaced apart from each other and disposed on opposite sides of the moisture sensor, each of the RFID tags electrically coupled to the moisture sensor and each of the RFID tags including a circuit that determines an amount of moisture that is associated with the moisture sensor, each of the RFID tags configured to respond to a first radio frequency signal with a second radio frequency signal that includes information about the amount of moisture.

2. The moisture sensor unit of claim 1, further comprising a diaper into which the passive moisture sensor and the two passive RFID tags are embedded, the diaper defining the environment, a first one of the two passive RFID tags disposed in a first portion of the diaper so that if a wearer of the diaper is facing upwardly, then the a first one of the two passive RFID tags will be in a position to be responsive to the first signal, the second one of the two passive RFID tags disposed in a second portion of the diaper, different from the first portion of the diaper, so that if the wearer of the diaper is facing downwardly, then the a second one of the two passive RFID tags will be in a position to be responsive to the first signal.

3. The moisture sensor unit of claim 1, wherein the passive moisture sensor comprises a variably capacitive element that has a capacitance that is a function of the amount of moisture.

4. The moisture sensing system of claim 3, wherein the variably capacitive element comprises:
(a) a first conductive strip with a first plurality of spaced apart conductive fingers extending transversely therefrom;
(b) a second conductive strip with a second plurality of spaced apart conductive fingers extending transversely therefrom, the second conductive strip disposed relative to the first conductive strip so that the second plurality of spaced apart conductive fingers are interleaved with the first plurality of spaced apart conductive fingers so as to define a non-conductive path between the first conductive strip and the second conductive strip; and
(c) a waterproof substrate enveloping at least the first conductive strip and the second conductive strip,
wherein a portion of the environment adjacent to the first conductive strip and the second conductive strip has a permittivity that is a function of the amount of moisture.

5. The moisture sensor unit of claim 1, further comprising a flexible waterproof substrate that envelops the passive moisture sensor and the two RFID tags.

6. The moisture sensor unit of claim 1, wherein the two passive RFID tags include an energy harvesting circuit configured to harvest energy from the first signal.

7. The moisture sensor unit of claim 1, wherein the two passive RFID tags include:
(a) a digital circuit configured to generate a digital representation of the amount of moisture; and
(b) a modulating circuit configured to modulate the digital representation on to the second signal.

8. The moisture sensing system of claim 7, wherein the modulating circuit is configured to represent different data values with corresponding different phases in the second signal.

9. A sensing system for sensing moisture in an environment, comprising:
(a) a radio frequency transceiver configured to transmit a first signal and to receive a second signal;
(b) a passive moisture sensor associated with the moisture; and
(c) two passive RFID tags that are spaced apart from each other and disposed on opposite sides of the moisture sensor, each of the RFID tags electrically coupled to the moisture sensor and including a circuit that determines an amount of moisture that is associated with the moisture sensor, each of the RFID tags configured to respond to the first signal with the second signal so that the second signal includes information about the amount of moisture.

10. The moisture sensing system of claim 9, further comprising a diaper into which the passive moisture sensor and the two passive RFID tags are embedded, the diaper defining the environment, a first one of the two passive RFID tags disposed in a first portion of the diaper so that if a wearer of the diaper is facing upwardly, then the a first one of the two passive RFID tags will be in a position to be responsive to the first signal, the second one of the two passive RFID tags disposed in a second portion of the diaper, different from the first portion of the diaper, so that if the wearer of the diaper is facing downwardly, then the a second one of the two passive RFID tags will be in a position to be responsive to the first signal.

11. The moisture sensing system of claim 9, wherein the passive moisture sensor comprises a variably capacitive element that has a capacitance that is a function of the amount of moisture.

12. The moisture sensing system of claim 11, wherein the variably capacitive element comprises:
(a) a first conductive strip with a first plurality of spaced apart conductive fingers extending transversely therefrom;
(b) a second conductive strip with a second plurality of spaced apart conductive fingers extending transversely therefrom, the second conductive strip disposed relative to the first conductive strip so that the second plurality of spaced apart conductive fingers are interleaved with the first plurality of spaced apart conductive fingers so as to define a non-conductive path between the first conductive strip and the second conductive strip; and
(c) a waterproof substrate enveloping at least the first conductive strip and the second conductive strip,
wherein a portion of the environment adjacent to the first conductive strip and the second conductive strip has a permittivity that is a function of the amount of moisture.

13. The moisture sensing system of claim 9, further comprising a flexible waterproof substrate that envelops the passive moisture sensor and the two RFID tags.

14. The moisture sensing system of claim 9, wherein the two passive RFID tags include an energy harvesting circuit configured to harvest energy from the first signal.

15. The moisture sensing system of claim 9, wherein the two passive RFID tags include:
(a) a digital circuit configured to generate a digital representation of the amount of moisture; and
(b) a modulating circuit configured to modulate the digital representation on to the second signal.

16. The moisture sensing system of claim 15, wherein modulating circuit is configured to represent different data values with corresponding different phases in the second signal.

17. The moisture sensing system of claim 9, wherein the radio frequency transceiver is configured to assert an alarm signal when the amount of moisture exceeds a predetermined threshold.

18. The moisture sensing system of claim 17, further comprising a remote monitor, responsive to the radio frequency transceiver, configured to generate an audible alarm when the alarm signal is asserted.

19. The moisture sensing system of claim 17, wherein the radio frequency transceiver is further configured to transmit the alarm signal to a communications network and further comprising a device, in communication with the cellular network, that is configured to generate an alarm when the alarm signal is asserted.

20. A diaper for absorbing moisture voided by a wearer, comprising:
(a) an outer shell;
(b) an absorbent core structure disposed on the outer shell and configured to absorb moisture; and
(c) a moisture sensor unit coupled to the absorbent core structure, including:
(i) a passive moisture sensor disposed so that at least one electrical property of the moisture sensor depends on an amount of moisture in the absorbent core structure; and
(ii) two passive RFID tags that are spaced apart from each other and disposed on opposite sides of the moisture sensor, each of the RFID tags electrically coupled to the moisture sensor and including a circuit that determines the amount of moisture in the absorbent core structure, each of the RFID tags configured to respond to a first radio frequency signal with a second radio frequency signal that includes information about the amount of moisture, a first one of the two passive RFID tags disposed in a first portion of the diaper so that if a wearer of the diaper is facing upwardly, then the a first one of the two passive RFID tags will be in a position to be responsive to the first signal, the second one of the two passive RFID tags disposed in a second portion of the diaper, different from the first portion of the diaper, so that if the wearer of the diaper is facing downwardly, then the a second one of the two passive RFID tags will be in a position to be responsive to the first signal.
